(19) United States
(12) Patent Application Publication
(10) Pub. No.: US 2006/0101542 A1
(43) Pub. Date: May 11, 2006

(54) Insect resistance

(76) Inventor: Daniel F. Warnock, Savoy, IL (US)

Correspondence Address: GREENLEE WINNER AND SULLIVAN P C, 4875 PEARL EAST CIRCLE, SUITE 200, BOULDER, CO, 80301, US

(21) Appl. No.: 11/270,883

(22) Filed: Nov. 9, 2005

Related U.S. Application Data
(60) Provisional application No. 60/626,421, filed on Nov. 9, 2004.

(51) Int. Cl. A01H 5/00; A01H 1/00; C12N 15/82

(52) U.S. Cl. 800/279; 800/323

(57) ABSTRACT

The invention provides a method for establishing commercial cultivars of impatiens having elevated resistance to a specified insect pest. The resistance provided through use of the invention reduces physical damage to the plant caused by insect feeding, and secondarily reduces the likelihood of virus transfer to the plant via an insect vector. The invention is exemplified by breeding for resistance to Western Flower Thrips (WFT).

### CROSS REFERENCE TO RELATED APPLICATIONS

This application claims benefit of U.S. Provisional Application No. 60/626,421, filed Nov. 9, 2004, which is incorporated herein to the extent that there is no inconsistency with the present disclosure.

### ACKNOWLEDGMENT OF FEDERAL RESEARCH SUPPORT

This invention was made, at least in part, with funding from the United States Department of Agriculture CRIS Hatch Project ILLU-65-0308. The United States Government has certain rights in this invention.

### BACKGROUND OF THE INVENTION

The field of the invention is plant resistance to insect infestation. Exemplified herein is a breeding method to develop a population of impatiens having elevated resistance to feeding damage from western flower thrips.

Western flower thrips (WFT), Frankliniella occidentalis (Pergande), is an opportunistic insect pest in commercial greenhouses worldwide [Mound, L. A. et al. (1995) Thrips biology and management B. L. Parker, M. Skinner and T. Lewis (eds.) Plenum Press, New York p. 3-21]. Western flower thrips is a serious pest inflicting physical damage and vectoring both tomato spotted wilt virus (TSWV) and impatiens necrotic spot viruses (INSV) in many crops [Parrella, M. P. (1995) Thrips biology and management. B. L. Parker, M. Skinner and T. Lewis (eds.) Plenum Press, New York p. 357-363]. High-value greenhouse crops such as vegetables, fruit, and especially ornamentals are particularly vulnerable to economic losses associated with thrips damage due to the retail market requirements for an aesthetically pleasing product [Lewis, T. (1997) Thrips as crop pests. T, Lewis (ed.). CAB Intl., New York p. 1-15]. Western flower thrips feeding results in silver mottling or blotching, streaking, and distortion of the leaves and/or petals, all of which reduce the marketability of floriculture crops [Parrella, M. P. et al. (1987) Bul. Entomol. Soc. Amer. 33:28-34]. Managing thrips is difficult, once they have become established in a greenhouse, requiring floriculture crop producers to rely on insecticides for control. Thrips, however, have developed resistance to several insecticides, including abamectin, acephate, bendiocarb, bifenthrin, cypermethrin, diazinon, dimethoate, endosulfan, fenpropanate, methiocarb, methomyl, and permethrin, [Brødsgaard, H. F. (1994) J. Econ. Entomol. 87:1141-1146; Immaraju et al., (1992) J. Econ. Entomol. 85:9-14; Robb, K. L. et al., (1995) Thrips biology and management. B. L. Parker, M. Skinner and T. Lewis (eds.). p. 341-346; Zhao, G. et al., (1995) J. Econ. Entomol. 88:1164-1170]. Integrated pest management (IPM) practices, such as rotating insecticide classes, proper scouting with yellow sticky cards, and screening greenhouse vents, will slow the development of insecticide resistance by reducing the selection pressure of excessive insecticide applications on insect populations. Slowing the development of insecticide resistance is critical because emerging insecticides that effectively manage WFT are limited. Current IPM practices, while reducing insecticide applications, can be expensive (screening) and do not effectively control thrips (biological controls). Additional pest management options in IPM programs are needed.

Host plant resistance, a component of IPM programs, is a good control strategy for WFT [Mound et al. (1995) supra]. Suitability of host plants for thrips varies among genotypes within a plant species [Zeier, P. et al. (1995)Thrips biology and management. B. L. Parker, M. Skinner and T. Lewis (eds.) Plenum Press, New York p. 411-416]. A reduction in insect fitness due to host plant resistance is desirable in that insects not killed by plant allelochemicals will be more susceptible to insecticides and biological predators. Host plant resistance can increase the efficiency of control options available to crop producers and promotes longer useful life of currently available insecticides.

Results indicate that chrysanthemum (Dendranthema x grandiflorum Kitam), gladiolus (Gladiolus grandiflorus Linn.), and impatiens (Impatiens wallerana Hook. f) cultivars have varied levels of resistance to thrips [van Dijken, F. R. et al. (1995) Thrips biology and management. B. L. Parker, M. Skinner and T. Lewis (eds.) Plenum Press, New York p. 407-410; Zeier (1995) et al. supra; Herrin, B. B. et al. (2002) Hort Science 37:802-804 incorporated herein by reference]. For example, de Jager et al. (1995) J. Econ. Entomol. 88(6):1746-1753, found 6 of 10 chrysanthemum cultivars negatively impacted feeding of WFT larvae. The variation in cultivar resistance was related to compounds in the leaves.

General references in plant breeding include the following:
Callaway, D. J. and M. B. Callaway. 2000. Breeding Ornamental Plants. Timber Press Inc., Portland, Oreg. Pp. 323.
Fehr, W. R. 1987. Principles of cultivar development: Theory and technique. Vol. 1. Macmillan Publishing Company, New York, N.Y. Pp. 536.
Hallauer, A. R. and J. B. Miranda. 1981. Quantitative genetics in maize breeding. Ed. 2. Iowa State University Press, Ames, Iowa. Pp. 468.

Impatiens sales in the United States exceed $163 million making it the number one selling bedding plant (USDA, 2002). Due to a rapid crop cycle and high susceptibility to feeding by WFT, impatiens is a model crop for use in a host plant resistance breeding program. Host plant resistance to WFT feeding damage is known to be variable within impatiens cultivars [Herrin et al., (2002) supra]. Host plant resistance in impatiens limits the physical damage caused by WFT feeding and thereby indirectly can limit INSV spread. The mechanism(s) of resistance to feeding by WFT in impatiens, while unknown at present, can include physical, chemical, or a combination of both mechanisms. Physical mechanisms of resistance may include pubescence, flower color, pollen shed, and plant architecture. Chemical components associated with resistance can represent a broad array of chemical classes. Secondary metabolites have been found to contribute to thrips resistance in chrysanthemums [de Jager et al., (1995) supra]. Recent acquisitions of wild type impatiens populations provide researchers with the opportunity to broaden available resistance mechanisms.

The development of impatiens populations and elite breeding lines with resistance to feeding by WFT is desirable. Evaluation of impatiens germplasm is the first step toward the identification and development of germplasm with improved levels of resistance to thrips damage.

Inter-specific hybridization between Impatiens flaccida and Impatiens hawkeri was reported in U.S. Pat. No. 6,924,416. However, the majority of the F₁ plants were sterile and it was not possible to recover seed from self-pollination or backcrossing. The invention provides a method for establishing commercial cultivars of impatiens having elevated resistance to a specified insect pest. The resistance provided through use of the invention reduces physical damage to the plant caused by insect feeding, and secondarily reduces the likelihood of virus transfer to the plant via an insect vector. The invention is exemplified by breeding for resistance to Western Flower Thrips (WFT).

### SUMMARY OF THE INVENTION

The invention provides a method for establishing commercial cultivars of impatiens having elevated resistance to a specified insect pest. The resistance provided through use of the invention reduces physical damage to the plant caused by insect feeding, and secondarily reduces the likelihood of virus transfer to the plant via an insect vector. The invention is exemplified by breeding for resistance to Western Flower Thrips (WFT). Field observations have indicated resistance to Japanese beetle infestation in some cultivars, possibly the consequence of a common mechanism affecting resistance to both kinds of insect. Resistance to WFT can provide cross-over resistance to other species of thrips.

### BRIEF DESCRIPTION OF THE DRAWINGS

FIG. 1 is a pedigree diagram for two selected propagules 04-IL-1408 and 04-IL-1426. The sequence of crossing and selection steps begins at the bottom of the figure, with San Vito O. P. (open pollinated) Population 1 and San Vito O. P. Population 2, as described.

FIG. 2 is a bar graph showing frequency distributions of thrips resistance ratings (horizontal axis) of original San Vito Populations, (Cajun Carmine), previously identified as having some WFT resistance and of a selected population of the 04-IL-1000 and 05-IL-100 generations, obtained as diagramed in FIG. 1. Data for plants rated 9 (9 and higher) were omitted.

### DETAILED DESCRIPTION OF THE INVENTION

All words and phrases relating to the fields of plant breeding, plant genetics and horticulture are intended herein to have their commonly accepted meaning as understood by those skilled in their respective fields. For convenience certain terms are herein described, without intent to limit or alter their accepted meaning in the art.

The term “impatiens” as used herein refers to any species of impatiens. All genotypes specifically described herein belong to the impatiens.

“Insect resistance” refers to the ability to avoid damage to a plant resulting from contact with a specified insect pest. Resistance can be measured in several ways, often by a quantitative or semi-quantitative measurement of damage observed under controlled exposure of the plant to a specified insect pest under conditions where other environmental factors are kept constant. Low insect damage can then be correlated with high insect resistance and plants can be ranked according to insect resistance, from highest to lowest. Heterozygous populations typically display a wider range of resistance than do homozygous populations. Nevertheless, the relative level of resistance in heterozygous populations can be evaluated by statistically analyzing the distribution of resistance over the population, where parameters such as average resistance, mean resistance, and peak resistance are shifted toward greater resistance (reduced damage) compared to other populations.

“Ornamental phenotype” refers to a trait or characteristic that imparts commercial value to a plant sold primarily as ornamental plants. Characteristics such as flower color, color intensity and color pattern, flower size, number of flowers per plant, length of time the plant blooms, plant height, growth habit, odor and leaf color are some of the many phenotypes which can be selected for in breeding plants. In the case of impatiens, ornamental varieties typically display low resistance to insect damage, especially to thrips. By use of the breeding method described herein, higher insect resistance can be introduced into ornamental varieties, by selecting for improved insect resistance and also selecting for desired ornamental phenotypes.

A “cross” is a product of cross-pollinating, (also termed “cross-breeding”) two parent plants. An “initial cross” in a breeding program is typically produced by cross-breeding parent plants that display variation for a given trait, such as insect resistance. In breeding for thrips resistance, as exemplified herein, an initial cross was made between a wild impatiens genotype from Costa Rica, having relatively high thrips resistance but lacking a desired ornamental phenotype, and a commercial cultivar that had a desired ornamental phenotype but low thrips resistance. All crosses described and claimed herein are intra-specific crosses.

“Selfing” refers to a process in which pollen from a single progeny plants of a cross is used to fertilize flowers of the same plant. “Sibing” refers to a process in which progeny sibling plants of a cross are fertilized to each other. “Crossing”, “cross-breeding” and “cross-fertilization” are terms used interchangeably to include any non-self fertilization breeding, including sibing and crossing with more distantly related plants. Selfing and sibing tend to result in some of the progeny plants being more homozygous for some genes than either parent. In the case of a quantitative trait such as insect resistance, many genes are likely to contribute to the amount of trait that is measurable.

In conventional plant breeding, greater homozygosity can be achieved by successive rounds of selfing, selecting the best progeny of each generation to be parents for the succeeding round. As described in detail herein, applying the conventional methods is not possible with impatiens because each successive round of self-fertilization results in a significant increase in male-sterility. In the method of the invention, selfing is carried out for not more than two successive generations. Additional non-self fertilization usually sibing must be used to re-establish male fertility. Although the added cross-fertilization step can reduce the homozygosity of insect resistance genes, it has the advantage of allowing the breeder to introduce other desired traits, e.g. ornamental phenotypes, for later selection.

It is possible to compensate for an overall increment of heterozygosity by strict selection, choosing only the most insect-resistant plants as parents for the next round of breeding. Such plants are likely to be the most homozygous for insect-resistance genes. The practice of this invention includes selecting from 2% to 10% of the plants having the highest insect resistance, as parent plants for subsequent crosses. A breeder can choose the most selective criteria (2%) or lease selective criteria (40%) or any intermediate level of selection desired, for example 5%, depending on the number of plants available and other factors which those skilled in the art may take into account.

The invention uses the variation in resistance to thrips that exists in both wild and commercial cultivars. Steps in the breeding process include:

A method of plant breeding to produce a strain of impatiens having a desired ornamental phenotype and reduced insect damage compared to a parent ornamental strain in the presence of a specified insect pest, comprising the steps of:

1) making an initial cross between representative individuals of a first genotype of an impatiens species, and a second genotype of the same species, the first genotype having relatively less insect damage compared to other genotypes in the presence of the specified insect pest, at least one individual of the cross typically having a desired ornamental phenotype;

2) quantitatively evaluating insect damage to individual seedling progeny plants of the cross in the presence of the specified insect pest;

3) selecting the seedling progeny plant having the least insect damage, the number of plants selected being at least 2% and not more than 10% of the total progeny plants;

4) selfing the plants selected in step (3) for 1 or 2 generations and repeating steps (2) and (3) for the progeny plants of each generation;

5) repeating steps (1-4) using a selected plant from the previous selection step as at least one parent in each cross until a desired mean level and uniformity of insect damage measured in total progeny is achieved; and

6) repeating steps (1-4) using an individual plant of step (5) as at least one parent and selecting additionally for a desired ornamental trait.

Since the resulting populations remain heterozygous, there will be variation among individual plants in the selected population. However, the mean level of resistance increases as steps of the process are further iterated, such that the mean level of resistance of the selected population exceeds the mean resistance level of either of the original parents. Additional rounds of crossing and selfing, combined with strong selection pressure can eventually result in homozygosity for the selected trait; however, homozygosity is not essential since an increase in the mean of resistance can be of significant commercial value in a horticultural plant. Once a plant of commercial value is identified it can be maintained as a cultivar by vegetative propagation.

When breeding floriculture plants, one must have a targeted phenotype and genotype in mind before the initial cross pollination event is performed. To exemplify the invention, the goal was to develop a commercially acceptable, yet novel, Impatiens wallerana (bedding plant impatiens) phenotype with improved resistance to the western flower thrips, a significant insect pest in production greenhouses. Most commercial impatiens cultivars are 12″ to 18″ tall with a spreading habit reaching 24″ in width upon maturity. Commercial cultivars typically are well branched with glossy dark green leaves. Flowers are large, profuse, and held above the foliage with intense and pure colors. With these traits in mind, the breeding program sought to obtain a large phenotype (greater than 18″ tall with more than a 24″ spread) with acceptable branching, leaf color, flower size, flower number, flower display, and flower colors. An additional goal was to incorporate resistance to western flower thrips feeding into these impatiens genotypes. During the process several barriers were encountered that had to be overcome. As such, the techniques developed by the inventor are specific for breeding of impatiens and selection for a quantitative trait such as insect resistance, exemplified by resistance to western flower thrips feeding.

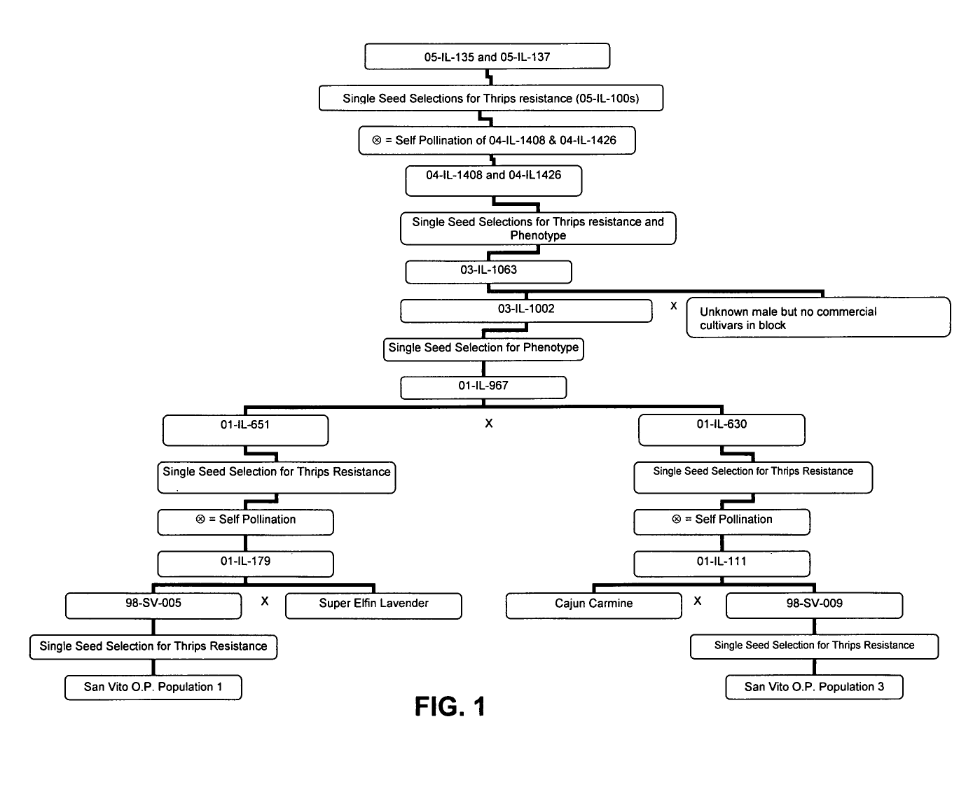

The efficient identification of crops with improved insect resistance levels depended on several components being available to researchers. Insects were needed in large quantities throughout the year. A method to quantitatively evaluate insect damage had to be established. Plants from several geographical areas or breeding programs needed to be available to provide useful genetic diversity. In the case of western flower thrips, the laboratory at the University of Illinois had successfully established and maintained a vigorous insect colony capable of supplying massive numbers of western flower thrips at any given time. The rearing protocols for this colony are modifications of rearing protocols from North Carolina State University and the University of California, Davis. These universities rear western flower thrips for insecticidal trails and viral transmission studies. A reliable and simple evaluation technique, based on the number of leaves expressing western flower thrips feeding damage after inoculation, was developed, utilized and described by Herrin et al. (2002) supra. This evaluation method has successfully been used to create more resistant impatiens genotypes based on breeding methods described herein and exemplified in the diagram of FIG. 1. It will be understood that quantitative methods of evaluating insect damage will vary depending on the insect species and its manner of inflicting damage. Those skilled in the art will be familiar with, or readily able to devise, such methods.

The selection of increased resistance to feeding by western flower thrips was only possible because a diverse group of germplasm was available for evaluation. Warnock, D. (2003) HortScience 38(7):1424-1427, incorporated herein by reference, described the diversity of impatiens populations collected from Costa Rica, while Herrin, B. B. et al. (2002) supra determined that some commercial cultivars of impatiens had varying levels of resistance. Therefore, the potential for improving resistance to western flower thrips feeding existed within available germplasm and the tools necessary for proper evaluations were developed by the inventor.

To make significant genetic gain in impatiens, breeders must overcome some limitations that are unique in this crop species. Impatiens are naturally out-crossing species. Flowers are perfect, containing both male and female organs; however, temporal separation of pollen shed and stigma receptivity encourage cross pollination. In nature, this is achieved when insects transfer pollen from one plant to another. Cross pollination ensures heterozygous plants readily exist in the population and thereby maintains genetic diversity. From a survival standpoint, this is a good strategy for survival in multiple environments as plants can adapt to new situations with ease. From a plant breeding perspective, heterozygous plants are not as desirable.

In a conventional plant breeding program, researchers strive to create homozygous plants (often called inbreds) that contain specific desirable traits. Homozygous plants having various characteristics are then cross pollinated with one another to create progeny containing multiple desirable traits. Impatiens create a specific challenge to plant breeders in that as homozygosity increases in a plant line, pollen production rapidly decreases to a point that the lines are no longer useful. Male sterility appears after one or two generations when self pollination is used to generate more homozygous lines. As such, true inbred lines can not exist in impatiens breeding programs. Impatiens “inbred lines” are actually populations derived from a single seed source. This aspect of impatiens biology is important because most traditional plant breeding models are based upon homozygous parent lines cross-bred to create hybrid progeny. Lacking homozygous lines, it becomes difficult to determine inheritance patterns for specific traits of impatiens based on conventional plant breeding models. Most improvements in impatiens are indicated by shifts over several generations in mean values of quantitative traits towards desirable phenotypes and genotypes.

From a commercial standpoint, most companies seek impatiens genotypes that are phenotypically stable across multiple environments. Branching habit, leaf color, flower size, flower number, and flower color purity must be consistent for an impatiens cultivar to succeed commercially. This fact necessitates evaluating potential releases in multiple locations and seasons. Once the more difficult traits, such as resistance to disease or insects, are stabilized in a germplasm source, selection for the desired horticultural traits can begin. The breeding method of the present invention is exemplified with intra-specific crossing of Impatiens wallerana Hook.f. cultivars. Other species of Impatiens can be improved using intra-specific crosses according to the described breeding methods.

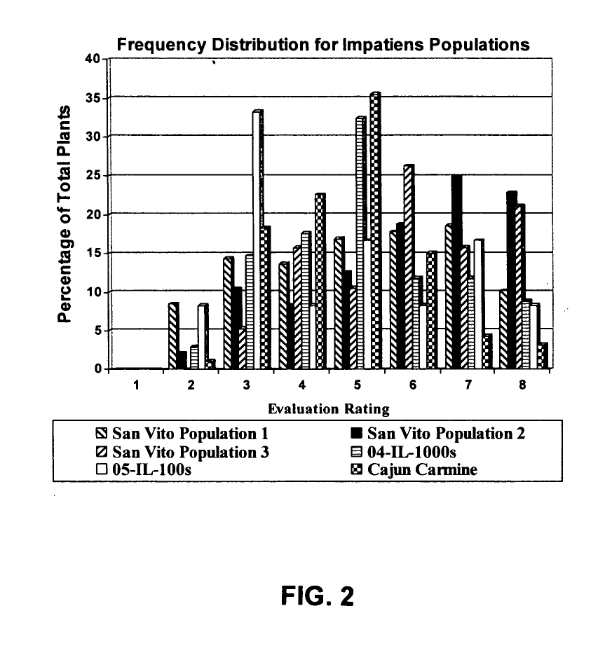

The breeding method of the present invention follows an annual cycle where seedlings are evaluated in the spring for resistance to insects such as western flower thrips. Seedlings with resistance are transplanted to a field environment during the summer where they are evaluated for the other desirable phenotypic traits described above. Seedlings passing these two evaluations are then transferred to the greenhouse during late summer or early fall where they are selectively pollinated to obtain the next generations for evaluation the following spring. Typically 800 seedlings are reduced to the best 16 plants during this process. This equates to 2% of the original population being used as parents for the next generation. Using this selection pressure and evaluation processes, gains in resistance to western flower thrips feeding have been obtained (FIG. 2) while maintaining other desirable phenotypic characteristics. The germplasm lines selected to date have shown an improved level of resistance to western flower thrips feeding as indicated by an improved population mean and shift in the overall trend towards lower feeding damage ratings. There remained a significant number of seedlings that had ratings of 9 on the 1 to 9 scale, in part due to the rating scale divisions. Classes below 9 are in 5 leaf intervals, while the 9 rating contains all plants with more than 35 damaged leaves. (See Table 1.) Therefore, a larger number of plants with a 9 rating appear in the data set than if the rating scale were indefinitely extended in 5 leaf intervals. Logistically an indefinitely extended rating scale is difficult to obtain. The 1 to 9 scale is an acceptable rating scale as resistant, moderately resistant, and susceptible genotypes are readily identified. The data for the current impatiens population indicates that the resistance to western flower thrips feeding is not homogenous at this time and that continued selection will result in greater genetic gain for resistance.

The breeding method just described is also applicable for selective breeding of other desirable traits in impatiens, including resistance to other insect pests, and is further applicable for the selective breeding of desired traits in other cross-pollinated, heterozygous plant species, including, for example cleome, salvia, and lobelia.

### EXAMPLE 1

### Evaluation of Resistance to WFT Feeding Damage

Stock plants of each genotype to be evaluated were grown from seed. Terminal cuttings were taken from the stock plants for vegetative propagation of each genotype to be tested and grown under conditions to prevent incidental exposure to insect pests found in greenhouses.

Nine plants of each impatiens genotype were transplanted daily into 12.7-cm (1.24 L) azalea pots filled with Sunshine Growing Mix (Sun Gro Horticulture, Inc., Bellevue, Wash.) to obtain a total of 324 plants. Plants were placed on greenhouse benches in a randomized complete block design arranged by transplant date. Immediately after transplanting, each plant was covered with a vented Plexiglas isolation cage [62 cm high and 12 cm diameter with 135 μm thrips screening (Greenthumb Group, Inc., Downer's Grove, Ill.) covering vents] to keep plants free of insects until inoculation with WFT. Pots containing the impatiens genotypes were placed on water collection trays for subirrigation and grown with day/night temperatures set at 24° C./18° C., respectively. Commercial production guidelines for impatiens [Corr, B. (1998) Ball redbook, 16^{th} ed. V. Ball (ed.) Ball Publ., Batavia, Ill] were followed except that no insecticides were applied and the fertilization rate was 300 mg·L⁻¹ N.

Seven to ten days after transplanting, plants were inoculated with ≈20 WFT adults and instars from a laboratory colony designed to maintain insect health and feeding aggressiveness [Steiner, M. Y. et al. (1998) Austral. J. Entomol. 37:106-106]. After inoculation, thrips were allowed to feed on individual plants for a 4-week period during which visual evaluations to estimate thrips feeding damage were conducted 0, 2, and 4 weeks after inoculation (WAI). Symptoms of WFT damage included leaf silvering or contorted growth due to feeding. The number of leaves exhibiting WFT injury was counted on each plant. Plants were assigned a 1 to 9 rating where a rating of 1 indicated no damaged leaves and a rating of 9 indicated the greatest number of damaged leaves (Table 1).

TABLE 1

| Rating | Number of damaged leaves |
|---|---|
| 1 | 0 |
| 2 | 1 to 5 |
| 3 | 6 to 10 |
| 4 | 11 to 15 |
| 5 | 16 to 20 |
| 6 | 21 to 25 |
| 7 | 26 to 30 |
| 8 | 31 to 35 |
| 9 | Greater than 35 |

Three researchers independently evaluated each plant. Ratings were pooled and a mean visual rating for each impatiens genotype within each block was calculated. Data were analyzed using Microsoft Excel 2000 spreadsheet software (Microsoft Corporation, Redmond, Wash.) and SAS System for Windows, release 6.12 (SAS Institute, Inc., Cary, N.C.). To normalize the data and allow appropriate statistical analysis to be conducted, the data were log transformed and analyzed as a randomized complete block design with sampling [Gomez, K. A. et al. (1984) Statistical procedures for agricultural reasearch. 2^{nd} ed. Wiley, New York]. This analysis is similar to a standard split-plot design with impatiens genotype as main-plot and weeks after inoculation as sub-plot treatments [Gomez, et a. (1984) supra].

### EXAMPLE 2

### Evaluation of Potential Parental Genotypes

Fifty-nine impatiens genotypes were grown from seed donated by Pan American Seed Co., collected from plants in three open pollinated populations near San Vito, Costa Rica. Population one plants have dark green leaves that were mottled with red pigmentation on the underside and red flowers. Population two consists of plants with uniform medium green foliage with white or orange flowers. Population three plants have light green foliage and lavender toned flowers. These populations represent wild type plants not previously selected for desirable commercial characteristics or resistance to insect pests. Mean visual ratings (Example 1) were determined four weeks after inoculation with 20 adult WFT per plant. Six genotypes having ratings <4.0 were selected for the breeding program. In addition, genotype No. 50 was included, since it was the only representative of population No. 3 with a rating close to 4.0, which was deemed the cutoff value for commercial acceptability.

The values observed for each genotype tested are given in Table 2.

TABLE 2

| Genotype | | | Visual Rating |
|---|---|---|---|
| Number | Population | n^{y} | (1 to 9)^{x} |
| 29 | 2 | 4 | 8.75 |
| 58 | 1 | 4 | 8.75 |
| 3 | 2 | 4 | 8.25 |
| 48 | 3 | 4 | 8.25 |
| 28 | 2 | 4 | 8.00 |
| 23 | 1 | 4 | 7.75 |
| 34 | 1 | 4 | 7.75 |
| 5 | 2 | 4 | 7.75 |
| 54 | 2 | 3 | 7.67 |
| 45 | 2 | 4 | 7.50 |
| 17 | 1 | 4 | 7.50 |
| 4 | 2 | 3 | 7.33 |
| 15 | 1 | 3 | 7.33 |
| 47 | 3 | 4 | 7.25 |
| 2 | 2 | 4 | 7.25 |
| 36 | 1 | 4 | 7.25 |
| 30 | 2 | 4 | 7.25 |
| 49 | 3 | 4 | 7.25 |
| 12 | 1 | 4 | 7.25 |
| 41 | 1 | 4 | 7.00 |
| 53 | 2 | 3 | 7.00 |
| 33 | 1 | 4 | 7.00 |
| 43 | 1 | 4 | 7.00 |
| 8 | 1 | 4 | 7.00 |
| 10 | 1 | 4 | 6.75 |
| 1 | 2 | 4 | 6.75 |
| 11 | 1 | 4 | 6.50 |
| 39 | 1 | 4 | 6.25 |
| 57 | 2 | 4 | 6.25 |
| 7 | 1 | 4 | 6.25 |
| 27 | 2 | 4 | 6.25 |
| 52 | 3 | 3 | 6.00 |
| 35 | 1 | 4 | 6.00 |
| 9 | 1 | 4 | 6.00 |
| 44 | 1 | 4 | 6.00 |
| 51 | 3 | 4 | 6.00 |
| 56 | 2 | 4 | 6.00 |
| 25 | 2 | 4 | 5.75 |
| 31 | 1 | 4 | 5.75 |
| 24 | 1 | 4 | 5.75 |
| 37 | 1 | 4 | 5.50 |
| 40 | 1 | 4 | 5.50 |
| 42 | 1 | 4 | 5.25 |
| 22 | 1 | 4 | 5.25 |
| 59 | 1 | 4 | 5.25 |
| 13 | 1 | 4 | 5.25 |
| 55 | 1 | 4 | 5.25 |
| 21 | 1 | 4 | 5.25 |
| 26 | 2 | 4 | 5.00 |
| 32 | 1 | 3 | 5.00 |
| 50 | 3 | 4 | 4.75 |
| 16 | 1 | 4 | 4.50 |
| 38 | 1 | 4 | 4.25 |
| 6 | 1 | 4 | 3.75 |
| 18 | 1 | 4 | 3.50 |
| 20 | 1 | 4 | 3.50 |
| 46 | 2 | 4 | 3.25 |
| 14 | 1 | 4 | 3.25 |
| 19 | 1 | 4 | 2.75 |

### EXAMPLE 3

In carrying out the breeding program generally described above, wild-type genotypes selected as described in Example 2 were crossed with a commercial cultivar previously identified as having some WFT resistance [Herrin et al. (2002) supra] A representative diagram is shown in FIG. 1. Following the breeding scheme diagrammed in FIG. 1, plants of the 04-IL-1000 and 05-IL-100 series, which represent the most recent selections of the breeding program, were compared with the original San Vito populations and a commercial cultivar (Cajun Carmine, Syngenta Seed Co., Downers Grove, Ill.) for WFT feeding resistance. The results are shown in FIG. 2. It can be seen that the mean rating for the 04-IL-1000 and 05-IL-100 plants has shifted to lower values compared to the San Vito populations, although a range of values is observed for all populations (FIG. 2 and Table 3). The mean of the most recent selections in the 05-IL-500 series is lower than those in the 04-IL-1000 series indicating genetic gain towards more resistant plants. When the most susceptible class (9.0 and greater) is excluded, it can be seen that the 04-IL-1000 and the 05-IL-100 populations were more normally distributed and shifted toward improved resistance (FIG. 2). Additionally, the frequency of plants with ratings considered commercially acceptable is increasing as the selection criteria are applied to impatiens populations (FIG. 2). Thus, a greater percentage of plants in the most recently selected populations have visual ratings ≦3.0 than the original San Vito Populations, improved populations or the commercial cultivar. The commercial cultivar, Cajun Carmine, was previously identified as a potential source for resistance to WFT [Herrin et al. (2002) supra] in that it is more resistant than most commercially available cultivars. The most recently selected population derived from the breeding program is more resistant to WFT than the commercial cultivar indicating the validity of the techniques described herein.

TABLE 3

| | | Mean Visual Rating |
|---|---|---|
| Population | n | (1 to 9) |
| San Vito Population 1 | 118 | 5.18 |
| San Vito Population 2 | 48 | 6.02 |
| San Vito Population 3 | 19 | 6.11 |
| 04-IL-1000s | 34 | 5.06 |
| 05-IL-100s | 27 | 4.51 |
| Cajun Carmine | 94 | 4.71 |

The most recent generation, designated 05-IL-100, was obtained by selfing 04-IL-1408 and 04-IL-1426, also shown in FIG. 2. The mean damage rating in the 05-IL-100 population was 4.08, with the highest percentage of plants having ratings of 3.0. These results demonstrate significant improvement over populations from prior generations, including those used in the initial crosses.

Field observations have indicated that some plants have notably fewer Japanese beetles per plant as well. Although resistance to Japanese beetle infestation was not a selected trait, it is possible that one or more genes involved in thrips resistance may confer resistance to this beetle infestation.

Additional field observations indicate the presence of attractive horticultural phenotypes, indicating such features are not lost during several generations of selection for insect resistance.

1. A method of breeding impatiens to reduce insect damage in the presence of a specified insect pest, comprising a plurality of cross-breeding and self-breeding steps, comprising further the steps of:
a) evaluating insect damage to progeny of a given breeding step; and
b) selecting from 2% to 10% of the progeny which display the lowest insect damage in the presence of the specified insect pest; and
c) using progeny selected in step (b) as parent plants for additional steps of cross-breeding and self-breeding, whereby impatiens having reduced insect damage in the presence of a specified insect pest are obtained.

2. The method of claim 1 wherein self-breeding is not carried out for more than two consecutive generations.

3. The method of claim 2 wherein the insect pest is thrips.

4. The method of claim 2 wherein the insect pest is Japanese beetle.

5. The method of claim 1 wherein each step of cross-breeding or self-breeding is conducted using progeny from each preceding step selected according to step (b).

6. The method of claim 5 wherein each step of cross-breeding is succeeded by at least one self-breeding step.

7. The method of claim 5 wherein step (b) is conducted by selecting a maximum of 2% of progeny of the preceding step.

8. The method of claim 5 wherein step (b) is conducted by selecting a maximum of 5% of progeny of the preceding step.

9. The method of claim 5 wherein step (b) is conducted by selecting a maximum of 10% of progeny of the preceding step.

10. The method of claim 5 wherein at least one cross-breeding step is carried out by sibing.

11. The method of claim 1 wherein step (b) includes additionally selecting for a second phenotype.

12. The method of claim 11 wherein the second phenotype is an ornamental trait.

13. A method of plant breeding to produce a strain of impatiens having a desired ornamental phenotype and reduced insect damage compared to a parent ornamental strain in the presence of a specified insect pest, comprising the steps of:
a) making an initial cross between an individual plant of a first genotype of an impatiens species, and a plant of a second genotype of the same species, the first genotype having relatively less insect damage compared to other genotypes in the presence of the specified insect pest, at least one individual of the cross having an ornamental phenotype;
b) quantitatively evaluating insect damage to individual seedling progeny plants of the cross in the presence of the specified insect pest;
c) selecting seedling progeny plants having the least insect damage, the number of plants selected being at least 2% and not more than 10% of the total progeny plants;
d) selfing the plants selected in step (c) for 1 or 2 generations and repeating steps (b) and (c) for the progeny plants of each generation;
e) repeating steps (a-d) using a selected plant from the previous selection step as at least one parent in each cross until a desired mean level and uniformity of insect damage measured in total progeny is achieved; and
f) repeating steps (a-d) using an individual plant of step (e) as at least one parent and selecting additionally for a desired ornamental trait.
